(19) United States
(12) Patent Application Publication
(10) Pub. No.: US 2021/0213048 A1
(43) Pub. Date: Jul. 15, 2021

(54) METHOD FOR DETECTION AND TREATMENT OF CANCER

(71) Applicant: Shui Yin Lo, Arcadia, CA (US)

(72) Inventor: Shui Yin Lo, Arcadia, CA (US)

(21) Appl. No.: 16/740,905

(22) Filed: Jan. 13, 2020

(51) Int. Cl. A61K 33/00; A61B 5/00; A61B 5/01; A61H 39/02

(52) U.S. Cl. CPC A61K 33/00; A61B 5/0075; A61B 5/0036; A61B 5/08; A61H 39/02; A61B 5/4312; A61B 5/015

(57) ABSTRACT

A method for detection and treatment of cancerous cells utilizes an infrared imaging device to capture a first infrared image of hot spots near a meridian pathway on the body. The hot spot may indicate irregularities in cells in the body. The meridian pathway acts as a channel to the interior of body, where cancerous cells may be active. A white spot indicates an irregularly hot cell, indicating potential cancer. A predetermined amount of solution that contains solid water particles is administered orally, intravenously or topically, every day for a long period of time.

### FIELD OF THE INVENTION

The present invention relates generally to a method for detection and treatment of cancers. More so, a method for detection and treatment of cancers utilizes an infrared imaging device for capturing a infrared image of at least two hot spot near acupoints or meridian pathway on the body to indicate irregularities in skin temperature; one hot spot is under the armpit one acupoint HT1, and the second hot spot is on a meridian that passes near an organ, which has cancer, and then administering a predetermined amount of solution that contains solid water daily for a long period of time.

### BRIEF DESCRIPTION OF THE DRAWINGS

The invention will now be described, by way of example, with reference to the accompanying drawings, in which:

FIGS. 1A, and 1B, illustrate thermograph images of a male with prostrate in excellent health, in accordance with an embodiment of the present invention;

FIGS. 2A, 2B, 2C, 2D, 2E, and 2F illustrate thermograph images of a male with early stage prostate cancer, in accordance with an embodiment of the present invention;

FIGS. 3A, 3B, 3C, 3D, 3E, and 3F illustrate thermograph images of potential breast cancer in a female, in accordance with an embodiment of the present invention;

FIGS. 4A, 4B, 4C, 4D, 4E, and 4F illustrate thermograph images of potential prostate cancer in a male, in accordance with an embodiment of the present invention;

FIGS. 5A, 5B, 5C, 5D, 5E, and 5 illustrate thermograph images of potential colon cancer in a male in his sixties, in accordance with an embodiment of the present invention.

Like reference numerals refer to like parts throughout the various views of the drawings.

### DETAILED DESCRIPTION OF THE INVENTION

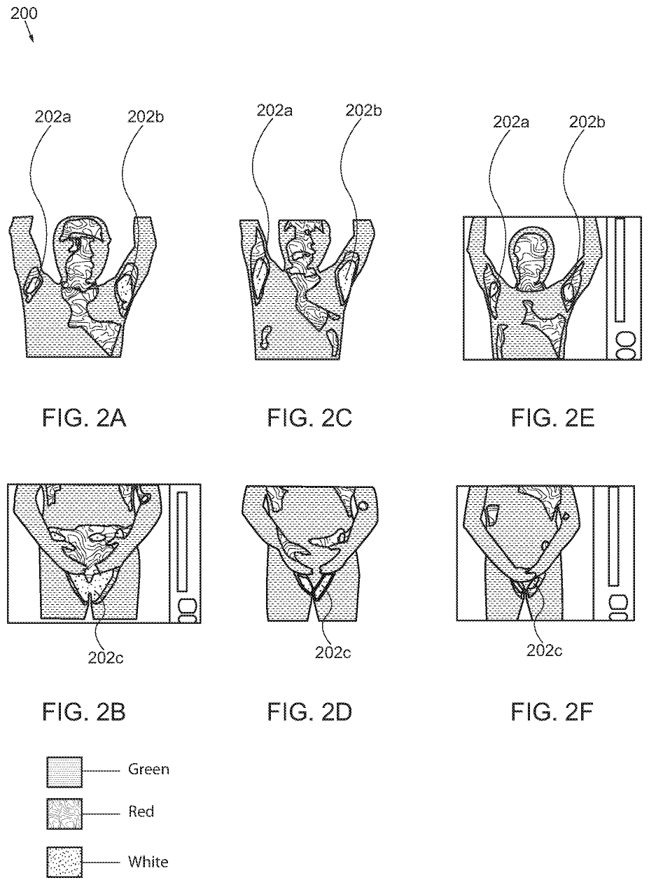

The following detailed description is merely exemplary in nature and is not intended to limit the described embodiments or the application and uses of the described embodiments. As used herein, the word “exemplary” or “illustrative” means “serving as an example, instance, or illustration.” Any implementation described herein as “exemplary” or “illustrative” is not necessarily to be construed as preferred or advantageous over other implementations. All of the implementations described below are exemplary implementations provided to enable persons skilled in the art to make or use the embodiments of the disclosure and are not intended to limit the scope of the disclosure, which is defined by the claims. For purposes of description herein, the terms “first,” “second,” “left,” “rear,” “right,” “front,” “vertical,” “horizontal,” and derivatives thereof shall relate to the invention as oriented in FIG. 2. Furthermore, there is no intention to be bound by any expressed or implied theory presented in the preceding technical field, background, brief summary or the following detailed description. It is also to be understood that the specific devices and processes illustrated in the attached drawings, and described in the following specification, are simply exemplary embodiments of the inventive concepts defined in the appended claims. Hence, specific dimensions and other physical characteristics relating to the embodiments disclosed herein are not to be considered as limiting, unless the claims expressly state otherwise.

It should be clearly understood that like reference numerals are intended to identify the same structural elements, portions, or surfaces consistently throughout the several drawing figures, as may be further described or explained by the entire written specification of which this detailed description is an integral part. The drawings are intended to be read together with the specification and are to be construed as a portion of the entire “written description” of this invention as required by 35 U.S.C. § 112.

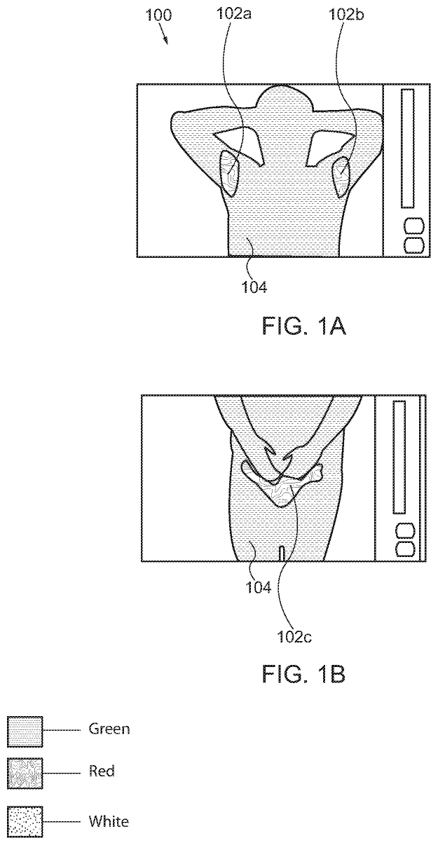

The present invention, referenced in FIGS. 1A-5F is directed to a method 100 for detection and treatment of cancer. The method 100-500 for detection and treatment of cancer utilizes an infrared imaging device for capturing a first infrared image of at least two hot spots near a meridian pathway on the body to indicate irregularities in skin temperature. The meridian pathway is based on a meridian theory of traditional Chinese medicine for identifying at least one acupoint along the meridian pathway. The higher body surface temperature indicated by the hot spot on the meridian pathway is thus, a reflection of the temperature of the internal organs where the meridian pathways pass through.

As measured by an infrared imaging device, hot spots and hotlines on the body surface may be an indication of potential cancer. In one embodiment in which a thermograph is used to capture the thermal image, the hot spot is indicated by a white spot, and cooler areas showing normal cells are indicated by a green region.

After identifying the abnormalities in skin temperature through the thermal imaging device, a predetermined amount of solid water particles is administered.

In another aspect, the at least one meridian pathway includes at least one member selected from the group consisting of: a bladder meridian, a stomach meridian, a gallbladder meridian, a large intestine meridian, a small intestine meridian, and a tripe heater meridian.

In another aspect, the infrared imaging device comprises a thermograph.

In another aspect, the step of administering a predetermined quantity of solid water particles includes delivering the predetermined quantity of solid water particles through at least one of the following: oral administration, topical administration, vapor administration, and intravenous administration.

In another aspect, the oral administration comprises a liquid, a pill, or a gel consisting of the predetermined quantity of solid water particles.

In another aspect, the topical administration comprises a cream consisting of the predetermined quantity of solid water particles.

In another aspect, the vapor administration comprises a nebulizer and an inhaler consisting of the predetermined quantity of solid water particles.

In another aspect, the intravenous administration comprises an intravenous injection consisting of the predetermined quantity of solid water particles.

In another aspect, the cream is configured to be applied at least topically to at least one member selected from the group consisting of: the left and right front center ears, left and right inner points of the eyes, left and right forehead temple, left and right collar bone, and left and right arm pits.

One objective of the present invention is to provide Chinese medicinal theories of the meridian for detection and treatment of cancerous cells in the prostate, breast, lung, and colon.

Another objective is to utilize a thermograph to identify abnormal skin temperatures.

Another objective is to administer solid water particles by a variety of routes of administration including: oral, intravenous, intramuscular, subcutaneous, intrathecal, topical, inhalation.

Yet another objective is to provide multiple delivery means for administering the solid water particles.

From the correlation of hundreds of images of different people, the present invention has deduced that the abnormal high temperature of skin temperature under the arm pit, where lymphatic node is, is related to a very active immune system. In one embodiment, the abnormal high temperature is detected by an infrared imaging device that displays the image as a thermograph. The very active system indicates that it is fighting cancers, if the person doesn't have fever from bacteria and virus infection.

Those skilled in the art will recognize that infrared imaging is non-invasive, and is a passive image device. Infrared imaging relies on the infrared emitted by the body. Thus, multiple images of the body, and especially near the meridian pathways may be captured without any harm to the subject. The images are then immediately available for viewing. Some advantages of using an infrared imaging device include, without limitation, the abnormally high temperature can be identified with simple training; the cost of the equipment is low; the sensitive imaging; and the easy viewing of early developing stage of cancers that other systems may not be able to detect.

Those skilled in the art will recognize that a thermograph is an instrument that produces a trace or image representing a record of the varying temperature or infrared radiation over an area or during a period of time. In one embodiment, the color code for the thermographs includes white the hottest, then red, yellow, green, blue, and black as the coldest. A green color on the thermograph is adjusted to be the color for normal skin temperature. For purposes of the present invention, abnormally high temperature is shown in white color, or the highest temperature of the body, which is about 3° Celsius higher than normal skin temperature. In one embodiment, if the excessive-high temperature which is in white color, also occurs in another area of well-known cancer places, such as breast, prostate, lungs, and colon, then there exists a potential for cancer, or cancer may have already developed.

As discussed above, the hot spot may be detected by utilizing an infrared imaging device, such as a thermograph, to capture a thermograph image on a portion of a body. The thermograph image may display at least one hot spot on the body. The hot spot may be indicative of an abnormality in skin temperature, which indicates cancer.

Those skilled in the art will recognize that cancer cells have a higher metabolism rate than normal cells and generate more heat; thus resulting in higher skin temperatures. Generally, the generated heat from cancerous cells is transmitted either via meridian channels inside the body to places where meridians are close to the surface or through connective tissues to the surface of the skin directly above the cancerous cells.

The method 100 may further comprise a Step 104 of identifying at least one meridian pathway, the at least one meridian pathway defined by a network that interconnects at least one internal organ. Those skilled in the art, in light of the present teachings, will recognize that the meridian pathway comprises a discrete, non-physical network that connects different internal organs of the body. The meridian pathway may also be defined as a line of energy flow from an internal organ to the surface of the body. Those skilled in the art will recognize that the meridian pathway is determined through traditional Chinese medicinal means.

It is also known that many aspects of traditional Chinese medicine are based on the meridian theory. Meridians are a discrete, non-physical network that connects different parts of the body together, namely internal organs. The meridian pathway creates a comprehensive map in the body that creates a channel for the organs to communicate and channel energy, or qi. Along the meridian path, there are acupuncture points.

The hot spot on the skin directly over the meridian pathway indicates a potentially cancerous cell, which may be symptomatic of a variety of cancers. This is because, as discussed above, the meridian pathway serves as an optical fiber that channels the heat of internal organs to the surface of the body. The higher body surface temperature indicated by the hot spot is thus, a reflection of the temperature of the internal organs where the meridian pathways pass through.

Specifically, the hot temperature along the meridian pathway is an indication of the excessive heat being generated by the internal organs. Those skilled in the art will recognize that internal organs emit hot infrared radiation, which are transmitted preferentially along the meridian pathways and peaks at known acupoints that are close to the surface of the body. In essence, internal organ abnormalities can be revealed by taking thermographs of the body and corresponding problematic organs with the meridian pathways.

In some embodiments, at least one hot spot is determined to locate approximately under the armpits where acupoint HT1 is at. The armpits are indicative of various cancers, including, without limitation, prostate cancer, breast cancer, lung cancer, and colon cancer. The next step is to determine if the at least a second hot spot is located approximately underneath the nipples around acupoint ST 18. This may indicate breast cancer.

Another step comprises determining the second hot spot which is located approximately underneath the testicles around acupoint RN1. This may indicate prostate cancer.

Another step comprises determining the second hot spot which is located approximately around the lung meridian and acupoint point LU2. This may indicate lung cancer.

Another step comprises determining the second hot spot which is located approximately across the belly. The hot spot may be displayed in the thermograph as a thick white line across the belly, which may be identified to the location of a special DAI meridian. This may indicate colon cancer.

The treatment of cancer comprises a steo of administering a predetermined quantity of solid water particles. In one possible embodiment, the mechanism of healing by solid water particles may be explained simply as follows: meridians are hypothesized to consist of solid water particles (or stable water clusters). The malfunction of organs along meridians is explained in Chinese medicine as the blocking of the flow of qi along the meridians. By drinking solid water particles, the charged solid water particles repair the meridian and unblock it, so that qi can freely flow again.

In one embodiment, the solution that contains solid water particle consists of only pure water molecules and no other chemicals and hence does not have harmful side effects. The solid water particle may include, without limitation, stable water clusters, and IE. Those skilled in the art will recognize that solid water particles may be referred to in different scientific names, such as stable water clusters and IE crystals.

The delivery of the solid water particles may be performed through at least one of the following: oral administration, topical administration, vapor administration, and intravenous administration. In some embodiments, the administration of the solid water particles may take various forms, including, without limitation: drinking solution that contains solid water particles; rubbing cream that contains solid water particles; breathing in mist that contains solid water particles; spraying fine drops that contain solid water waters on skin; administering drops using eye droppers that contain solid water particles into affected area; and intravenous injection of solutions that contain solid water particles. The daily dosage depends on the severity of cancer. The more severe cancer requires the more dosage of the solutions contains solid water particles.

Then, application of solid water particles, such as drinking a specific amount of water that contains large number of solid water particles. In one embodiment the large number could be more than three million solid water particles per 60 ml water that are larger than 0.1 micron.

In one embodiment, if the change of temperature in 15 minutes is hotter than shown in the first infrared image, the status of the cancerous cell is considered serious. Larger daily dosage of solid water particles may be administered. However, if the change of temperature in 15 minutes is cooler than indicated shown in the first infrared image, the status of the cancerous cell is considered to be not so serious, the dosage can be less. In any case, the larger the dosage of solid water particles, the quicker the recovery time.

When the temperature of the abnormally high skin temperature in the armpit area and the area where cancer shows up decreases, or the area of abnormal high temperature decreases, the cancer is considered shrinking. When the temperature of these two abnormal high temperature become normal, treatment of cancer is considered complete.

One exemplary application of the method 100 is illustrated in FIGS. 1A, and 1B, showing a thermograph image 100 of a male with a prostrate in excellent health. The red colored areas 102a, 102b, 102c under the testicles and in the armpit are about 2° Celsius warmer than the green colored areas 104 of the normal skin temperature elsewhere. This is considered normal, temperature.

Another exemplary application of the method 200 is illustrated in FIGS. 2A, 2B, F, showing a thermograph image 200 of a male with early-stage prostate cancer. It is known in the art that for prostate cancer, the only definitive diagnosis for cancer is by biopsy, which is an intrusive procedure that may lead to a spread of cancer cells. The infrared image 200 is not able to diagnose cancer, but it may detect its existence once it is found there is cancer. FIGS. 2A and 2B show a pair of white spots 202a, 202b under the armpits indicating the possibility of cancerous cells. FIG. 2B shows a white spot 202c under the testicles and the white spot 202c is close to acupoint RN1 and to the skin area nearest to the prostate where have a great probability to have cancer or have cancer developed already. This early detection of abnormally high temperature at the white spots 202c under the testicles, and near the prostate, indicates an abnormal high biochemical reaction is occurring in the prostate area. Such high biochemical reactions may mean abnormal growth of cells of the prostate. Abnormal growth of cells may mean the growth of cancer cells.

The heat of the prostate cancer will transmit to its neighbor where the network of meridians system is. The Meridian system will transmit the heat to the area underneath the testicles area around the acupoint RN1. In order to see the heat underneath the testicles the subject is asked to raise the two testicles as far high as possible to expose the region for pictures taken with infrared imaging system. It is significant to note that the cancer does not necessarily occur underneath the testicles. The heat generated by cancer, or the natural heating up of the cells near it may produce abnormal high skin temperature in area beneath the testicles

FIGS. 2A and 2B shows thermographs taken before drinking the solution having solid water particles (SWP). The treatment of cancer further comprises a step of drinking solution having solid water particles. FIGS. 2C and 2D show the thermographs taken 15 minutes later after drinking SWP. FIGS. 2E and 2F show the infrared pictures by daily drinking SWP for three weeks, wherein the white area 202a, 202b, and 202c are shrinking significantly. The white area 202a, 202b under the armpit is estimated to shrink more than a half. The white area 202c under the testicles is estimated to shrink 90%. Here, the prostate cancer will continue diminishing if drinking SWP continues.

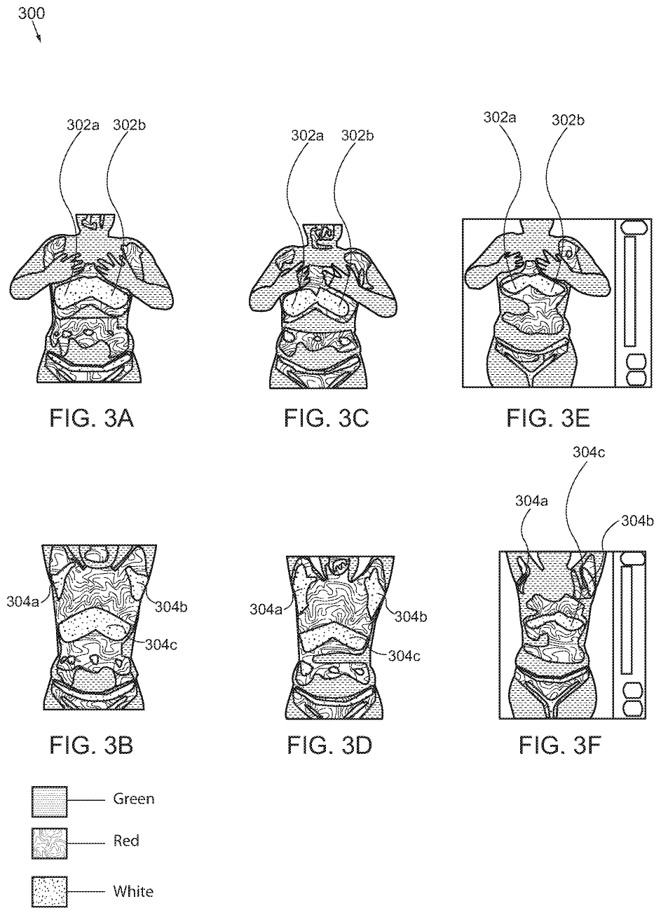

Another exemplary application of the method 300 is illustrated in FIGS. 3A, 3B, 3C, 3D, 3E, and 3F, showing a thermograph image 300 of potential breast cancer in a female. If the excessive high temperature which is shown in white color as a white spot 302a, 302b, occurs under the breast in addition to abnormally high temperature shown as a white spot 304a, 304b, 304c under the armpit and belly region, there is a great probability that it is a cancer, or a cancer is being developed. This early detection of abnormally high temperature under the breast indicates an abnormal high biochemical reaction is occurring in the breast. Such high biochemical reactions may mean abnormal growth of cells of the breasts. Abnormal growth of cells may mean the growth of cancer.

The thermographs in FIGS. 3A and 3B were captured before drinking water having the solid water particles (SWP). FIGS. 3A and 3B illustrate that the heat of the breast cancer transmits to a neighboring body area along with the network of meridians pathway. The Meridian pathway transmits the heat to the acupoints ST18 underneath the nipples and its surrounding area. The thermographs in FIGS. 3C and 3D were captured 15 minutes after drinking water having solid water particles (SWP). The inflammation under her armpits shown in white spots 302a, 302b are reduced in area. The thermographs in FIGS. 3E and 3F are captured 2 weeks later after daily drinking water having solid water particles (SWP) with one bottle (60 ml) per day. The right armpit white area are reduced by more than 90%, and the left white armpit area are reduced more than a half in two weeks. The white inflamed area under the two breasts are reduced in area by more than a half Inflammation under the breast was very abnormal. For people with perfect healthy breast conditions, there would be no white inflamed area under the breast.

In order to see the heat underneath the nipple the subject is asked to raise the two breasts with both hands upward to expose the region. The cancer does not necessarily occur underneath the nipples. The heat generated by cancers, or the natural heating up of the cells near it in the breast are sufficient to be seen by infrared images (or thermographs) underneath the nipples on and around ST 18. As a result of treatment with water having solid water particles (SWP), the female in the thermograph image 300 rated her breast 7 out of a grade system, 10 being the worse. Potential breast cancer is likely.

Thus, when the area under armpit and the area under breasts were both inflamed, the chance that tumor started to develop was quite high. The white spot 302a, 302b, 304a, 304b, 304c is 3° Celsius higher than normal green area. So inflammation of the breast was definite. The breast could not be regarded as normal. When drinking water having the solid water particle may reduce the inflammation area in two weeks by a large amount, it means that the health of the breast was improved by drinking water that contains solid water particles.

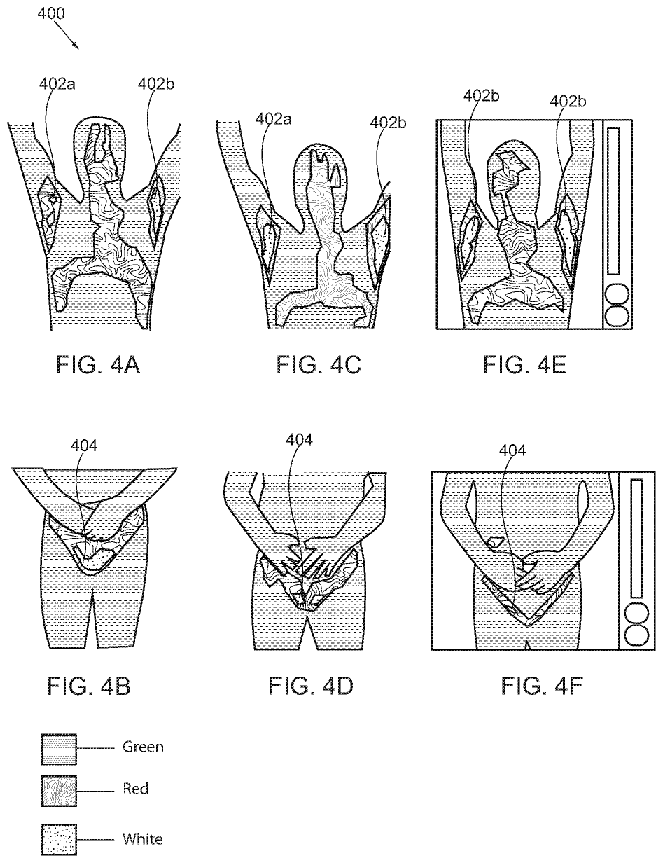

Another exemplary application of the method 500 is illustrated in FIGS. 4A, 4B, 4C, 4D, 4E, and 4F, showing a thermograph image 400 of potential prostate cancer in a male. Here, a male body is shown in his fifties. He knew has pre-cancer condition. His infrared image 400 is taken before water having solid water particles, as shown in FIGS. 4A and 4B. Here, large white spots 402a, 402b appear under the armpit, and a white spot 404 appears under the testicles, where acupoint RN1 is close by. FIGS. 4C and 4D illustrate the white spots 402a, 402b, 404 shrinking 15 minutes after drinking water having solid water particles. Looking now at FIGS. 4E and 4F, after drinking water having stable water clusters daily for two months, the infrared images 400 shows the white spots 402a, 402b may shrink a bit under the armpit, but the white spot 404 under the testicles almost disappear. His pre-cancer status of prostate may be removed.

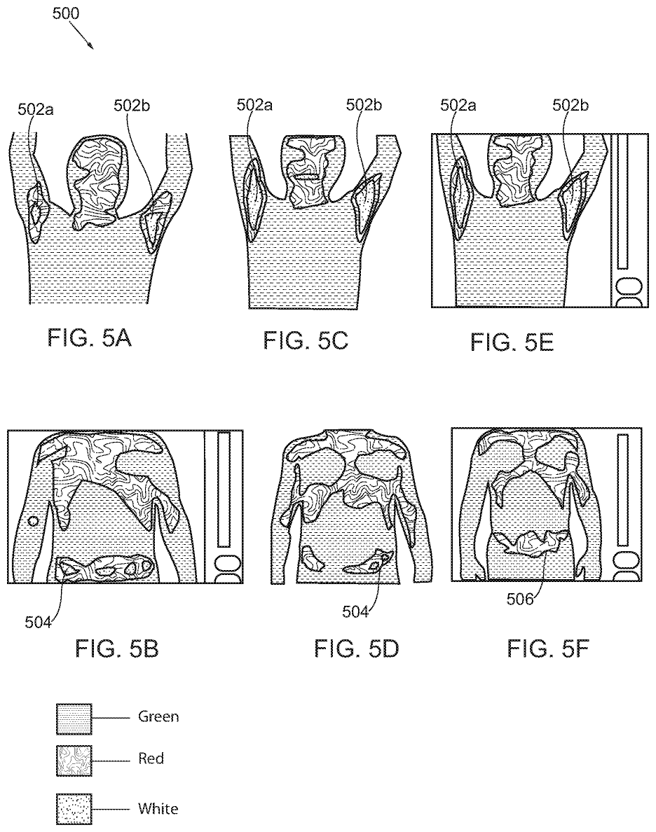

As shown in FIGS. 5A-5F, another exemplary application of the method 500 shows 500 thermograph images of potential colon cancer in a male in his sixties. Here, the infrared images 500 show FIGS. 5A and 5B captured before drinking water having solid water particles, they show large white spots 502a, 502b under the armpit, and many isolated white area across the belly 504 where underneath is the Dai Meridian having colon. FIGS. 5C and 5D show the white spots under the armpit 502a, 502b increases in area, and the white spot 504 in the colon area decreases. After drinking daily water having solid water particles for three weeks the white spot 504 of the colon completely disappears, and the white spots under the armpit 502a, 505b also decreases substantially, leaving only a green region 506 that indicates a normal temperature (FIGS. 5D and 5F). Thus, the possibility for the male to develop colon cancer is reduced close to zero if he continues drink water having solid water particles.

Other systems, devices, methods, features, and advantages will be or become apparent to one with skill in the art upon examination of the following drawings and detailed description. It is intended that all such additional systems, methods, features, and advantages be included within this description, be within the scope of the present disclosure, and be protected by the accompanying claims and drawings.

Since many modifications, variations, and changes in detail can be made to the described preferred embodiments of the invention, it is intended that all matters in the foregoing description and shown in the accompanying drawings be interpreted as illustrative and not in a limiting sense. Thus, the scope of the invention should be determined by the appended claims and their legal equivalence.

1. A method for detection and treatment of cancer, comprising:
capturing a plurality of infrared images of the body to determine at least one hot spot indicative of a high skin temperature approximately under the armpits where acupoint HT 1 is located;
determining at least one second hot spot on acupoint along one meridian;
administrating a sufficient large quality of solid water particles to the human body;
repeatedly administrating the sufficient large quality of solid water particles to the human body daily and many times a day; wherein
the said meridian passes through an internal organ which is affected by the cancer.

2. The method of claim 1, wherein the said second hot spot is located approximately underneath the nipples around acupoint ST 18 where the cancer is breast cancer.

3. The method of claim 1, wherein the said second hot spot is located approximately around the lung meridian and acupoint point LU where the cancer is lung cancer.

4. The method of claim 1, wherein the said second hot spot is located approximately underneath the testicles around acupoint RN 1 where the cancer is prostate cancer.

5. The method of claim 1, wherein the at least one hot spot is located approximately across the belly where the cancer is large intestine cancer having colon cancer.

6. The method of claim 1, further comprising a step of determining a larger size of hot spot with a higher temperature, wherein the larger size of hot spot represents as the more serious of cancer, wherein the higher temperature of hot spots represent the more serious of caner.

7. The method of claim 6, wherein the size of the hot spot is bigger than the size of the thumbnail, and the higher temperature of the hot spot is 3° C. above the normal skin temperature of the body.

8. The method of claim 6, wherein the administrating step can be selected from a group consisted of: orally administrating the sufficient large quality of solid water particles to the human body.

9. The method of claim 8, wherein the oral administration comprises a liquid, a pill, or a gel consisting of the predetermined quantity of solid water particles.

10. The method of claim 8, wherein the topical administration comprises a cream consisting of the predetermined quantity of solid water particles.

11. The method of claim 9, wherein the vapor administration comprises a nebulizer, a sprayer, or an inhaler consisting of the predetermined quantity of solid water particles.

12. The method of claim 10, wherein the intravenous administration comprises an intravenous injection consisting of the predetermined quantity of solid water particles.

13. The method of claim 9, further comprising a step of combining with other methods of treating cancers such as qigong, acupuncture, herbs, drugs in chemotherapy, surgery, or radiation therapy.
